(19) United States
(12) Patent Application Publication
(10) Pub. No.: US 2024/0252775 A1
(43) Pub. Date: Aug. 1, 2024

(54) Intubation device

(71) Applicant: aiEndoscopic AG, Zürich (CH)

(72) Inventors: David Gage, Thalwil (CH); Philippe Ganz, Zürich (CH)

(21) Appl. No.: 18/561,372

(22) PCT Filed: Apr. 5, 2022
(86) PCT No.: PCT/EP2022/059034

§ 371 Date: Nov. 16, 2023

(30) Foreign Application Priority Data
May 17, 2021 (EP) 21174057.6

(51) Int. Cl. A61M 16/04; A61M 16/00

(52) U.S. Cl. CPC A61M 16/0488; A61M 16/0003; A61M 16/0051; A61M 16/024; A61M 16/0418; A61M 2205/10; A61M 2205/18; A61M 2205/3306; A61M 2205/3553; A61M 2205/505; A61M 2205/581; A61M 2205/583; A61M 2209/01; A61M 2209/082; A61M 2210/1032

(57) ABSTRACT

The present invention relates to an intubation device for inserting an endotracheal tube into a patient's trachea. The intubation device comprises a laryngoscope blade for providing access to a pharyngeal cavity of the patient and a guide member which is substantially rigid and is curved in at least a section thereof. The intubation device further comprises a sled configured to be coupled to the endotracheal tube and to advance the endotracheal tube into the trachea of the patient and a drive device configured to drive the sled in an advancing direction. The sled is movably coupled to the guide member such that the sled is translationally movable along the guide member relative to the guide member in the advancing direction. The present invention further relates to an intubation device for providing assistance in delivering an endotracheal tube to a patient's trachea.

### CROSS REFERENCE TO RELATED APPLICATION(S)

This application is a Section 371 National Stage Application of International Application No. PCT/EP2022/059034, filed on Apr. 5, 2022, entitled “Intubation device”, which published as WIPO Publication No. 2022/242951 A1, on Nov. 24, 2022, in English, which claims priority to European Patent Application No. 21174057.6, filed on May 17, 2021, the contents of which are incorporated herein by reference in their entireties.

### BACKGROUND

Tracheal intubation is considered to be the worldwide gold standard to secure the airway of a patient in need of respiratory assistance. Tracheal intubation may be employed, for instance, for general anesthesia and/or in emergency medical services (EMS) and/or during intensive therapy.

In the most common state-of-the-art approach for performing a tracheal intubation, an endotracheal tube is placed into the trachea of a patient by a user while depressing the tongue with a laryngoscope and obtaining a direct line of vision onto the glottis of the patient, which forms the entrance of the trachea. Such an approach is referred to as direct-laryngoscopy.

In a first step of a process of placing the endotracheal tube into the patient's trachea by means of a laryngoscope, a laryngoscope blade of the laryngoscope may be inserted into the pharyngeal cavity of the patient. Generally, a distal end of the laryngoscope blade may be placed at or behind the epiglottis, with respect to a direction of insertion of the laryngoscope blade into the patient's pharyngeal cavity, in order to lift the pharyngeal anatomy of the patient to allow the endotracheal tube to be inserted into the patient's trachea. However, the correct/required placement of the laryngoscope blade may vary, e.g., depending on the configuration, e.g., the dimension(s) and/or the shape, of the laryngoscope being employed.

If the laryngoscope blade is placed too deep into the patient's body, the laryngoscope blade may obstruct the opening of the patient's trachea which may prevent, or at least impede, insertion of the endotracheal tube into the patient's trachea. Placing the laryngoscope blade not deep enough into the patient's body may prevent, or at least impede, the user's ability to open the patient's pharyngeal cavity which may also prevent, or at least impede, insertion of the endotracheal tube into the patient's trachea.

Once the laryngoscope blade has been placed correctly, e.g., when the distal end of the laryngoscope blade has been positioned at or behind the epiglottis, the user may then apply a force to the laryngoscope, more specifically to the laryngoscope blade, preferably substantially against the patient's tongue, to open the pharyngeal cavity of the patient. Once the patient's pharyngeal cavity has been opened, the user may then proceed by inserting the endotracheal tube into the patient's trachea.

Inability to perform tracheal intubation in time and/or misplacement of the endotracheal tube during the tracheal intubation procedure may result in severe morbidity or even mortality caused by asphyxia, hypoxemia, or pulmonary aspiration. Moreover, applying an incorrect amount of force to the laryngoscope, more specifically to the laryngoscope blade, e.g., by applying too much force, and/or misplacing the laryngoscope, more specifically the laryngoscope blade, in a faulty position and/or a faulty orientation, during the intubation process can lead to further morbidities, including, but not limited to, contusions, lacerations, and/or dental trauma. Casualties occur most frequently in EMS situations where tracheal intubation is performed under difficult and stressful circumstances.

However, anatomical factors or systemic conditions may make tracheal intubation difficult even for an experienced user/practitioner in a hospital setting.

A correct placement of the laryngoscope blade in the patient's pharyngeal cavity and/or a correct application of force to the laryngoscope, more specifically to the laryngoscope blade, to open the patient's pharyngeal cavity are skills which are generally developed by the user through extensive training and/or real-world experience. An experienced user may be able to determine the correct placement of the laryngoscope, in particular the laryngoscope blade of the laryngoscope, and open the patient's pharyngeal cavity, e.g., by means of visual and/or tactile feedback. Less experienced users, however, may commonly incorrectly place the laryngoscope in the pharyngeal cavity of the patient and/or incorrectly apply a force, e.g., by applying an incorrect amount of force, e.g., by applying too much force, and/or by applying a force in a wrong direction, during the intubation process. Moreover, even experienced users may misplace the laryngoscope in the pharyngeal cavity of the patient and/or may incorrectly apply a force and/or may apply a force in a wrong direction during the intubation process, e.g., due to human error and/or difficult and stressful circumstances and/or anatomical factors and/or systemic conditions, as described above.

Thus, alternatives to attempt to alleviate at least some of the difficulties of direct-laryngoscopic tracheal intubation have been developed, which include the use of video-laryngoscopes providing an indirect line of vision onto the glottis of the patient, referred to as indirect-laryngoscopy, flexible endoscopes to guide the endotracheal tube into the trachea of the patient and intubation devices which do not solely rely on manual guidance of the endotracheal tube.

Indirect-laryngoscopy devices, i.e., video-laryngoscopes, known from the prior art generally include a laryngoscope blade which has a camera mounted on a tip thereof. The camera may capture a view of the pharyngeal cavity of the patient during the intubation procedure in order to provide an image of the patient's anatomy, including one or more of the patient's tongue, uvula, epiglottis, corniculate cartilage, esophagus, vocal fold, and trachea, to the user. The camera may also provide an image of the distal end of the laryngoscope blade to the user.

US 2017/0304572 A1 discloses intubation devices in which a swing arm is configured to rotate about an axis via an electro-mechanical device or user actuation to deliver an endotracheal tube into a patient's trachea. The intubation devices further include a rail system for guiding the swing arm and the insertion of the endotracheal tube into the patient's trachea.

Moreover, US 2020/0178786 A1 also discloses an intubation device comprising a translation assembly having an endotracheal tube carriage for generating the insertion of an endotracheal tube into a patient's trachea. The endotracheal tube carriage is coupled to a lead screw of the translation assembly. The lead screw is driven by an electric motor, such that the endotracheal tube carriage is moved linearly along the longitudinal axis of the lead screw to advance the endotracheal tube. The endotracheal tube carriage is constrained by a linear guide. The endotracheal tube is positioned in a tube channel of a curved primary blade and guided therein into the patient's trachea.

However, disadvantages remain in the prior art which have not been addressed.

For instance, the devices known from the prior art are bulky and difficult to handle and to operate. Thus, said devices often require simultaneous cooperation of several users to handle and operate the devices in order to properly perform a tracheal intubation procedure on the patient.

This can lead to long tracheal intubation procedure times, failure to complete the tracheal intubation procedure and/or mistakes during the tracheal intubation procedure, such as misplacement of the endotracheal tube.

Furthermore, it is difficult or even impossible for a single user to perform a tracheal intubation procedure using the devices known from the prior art, in case no further persons are available.

Moreover, video-laryngoscopes which are known from the prior art and which may provide assistance to the user via a visual image of anatomical features of the patient and/or of the distal end of the laryngoscope blade to the user, may still be prone to misplacement of the laryngoscope in the pharyngeal cavity of the patient and/or an incorrect application of force as described above, e.g., due to human error and/or difficult and stressful circumstances and/or anatomical factors and/or systemic conditions.

Any of the above-identified situations may lead to severe morbidity or even mortality of the patient.

Moreover, the devices known from the prior art have complex constructions. For instance, replacing and/or servicing components of the devices may be difficult to perform.

### SUMMARY

It is therefore an object of the present invention to provide an intubation device with an improved means for delivering an endotracheal tube into a patient's trachea.

This object is achieved by an intubation device for delivering an endotracheal tube to a trachea of a patient according to a first aspect of the present disclosure, as defined by the features of claim 1. Variations and further developments are defined by the features of the dependent claims.

Preferably, the intubation device is electrically driven, preferably via at least one electric motor configured to drive at least one element of the intubation device, e.g., a movable advancing member configured to advance and at least partially deliver the endotracheal tube to the patient's trachea.

Preferably, the intubation device is a mobile device, which can be spatially moved to a desired location by a user.

Preferably, the intubation device is a robotic device, preferably configured to be operated autonomously, i.e. without user intervention or with minimal user intervention, or at least semi-autonomously for at least set time periods.

Preferably, the intubation device may be operated at least partially automatically and/or at least partially manually.

For instance, the intubation device may be configured such that the intubation device may be operated in a manual mode via manual input from a user and in an automatic mode, without or at least with reduced manual input from the user compared with the manual mode, sequentially and/or simultaneously.

Instead of or in addition to manual input, acoustic and/or written instructions on the display may be provided by the user (in a manual mode, in a semi-automatic mode, or in order to activate or deactivate a fully automatic mode).

For instance, in the case of operating in a manual mode and in an automatic mode sequentially, the intubation device may have a control configured to allow the user to switch the intubation device from the manual mode to the automatic mode and/or vice versa.

In the case of operating in a manual mode and in an automatic mode simultaneously, the intubation device may be configured to allow the user to operate the intubation device within certain set virtual boundaries, e.g., virtual spatial boundaries, wherein the intubation device may be configured to override the user's manual operation if the user crosses the virtual boundaries. This may help prevent or at least reduce the risk of injury to the patient caused by human error while allowing the user at least some freedom to operate.

The intubation device may be configured to provide feedback, e.g., haptic and/or visual and/or acoustic feedback, to the user when the virtual boundaries are crossed and/or just prior to the virtual boundaries being crossed, e.g., when a section of the intubation device or the endotracheal tube is within a certain distance from a portion of the anatomy of the patient.

For instance, the intubation device may be configured to vibrate, as a form of haptic feedback, to inform and/or warn the user that the virtual boundaries have been crossed and/or when the user attempts to cross the virtual boundaries and/or just prior to the virtual boundaries being crossed. For instance, a handle of the intubation device, which the user may grip while performing the tracheal intubation procedure, may vibrate as a form of haptic feedback, to inform and/or warn the user that the virtual boundaries have been crossed and/or when the user attempts to cross the virtual boundaries.

Moreover, the intubation device may be configured to provide an acoustic warning, as a form of feedback, to inform and/or warn the user that the virtual boundaries have been crossed and/or when the user attempts to cross the virtual boundaries and/or just prior to the virtual boundaries being crossed.

Moreover, the intubation device may be configured to provide acoustic and/or visual instructions to the user. Thus, the intubation device may provide instructions to the user so that the user may perform the tracheal intubation procedure manually but with audible and/or visible assistance of the intubation device, e.g., based on data sensed by the intubation device, instead of or in addition to performing the tracheal intubation procedure automatically or semi-automatically. Moreover, the intubation device may be configured to give instructions to the user for physically manipulating the intubation device, e.g., instructing the user to reposition the intubation device or at least a part or section thereof when it detects a misplacement of the intubation device or at least a part or section thereof.

The intubation device preferably comprises at least one laryngoscope blade for providing access to a pharyngeal cavity of the patient. The laryngoscope blade may be configured to depress the tongue of the patient in order to open the pharyngeal cavity.

The laryngoscope blade may be curved in at least a section thereof. This may facilitate the delivery of the endotracheal tube to the patient's trachea. The curvature of the laryngoscope blade may be determined based on the anatomy of the patient, for instance, based on size and/or age and/or gender of the patient. Thus, a variety of laryngoscope blades having different curvatures and/or other different geometric properties may be provided in order to adapt the shape and/or size of the laryngoscope blade to different patients having different anatomies.

Different laryngoscope blades may be attached to and released from the intubation device (e.g. from the handle of the device) in a modular manner, for instance, via a coupling mechanism, preferably a quick release coupling mechanism, such as a bayonet coupling.

The laryngoscope blade may define a channel through at least a section of the laryngoscope blade, the channel being configured to at least partially receive the endotracheal tube.

The laryngoscope blade may be fitted with a light source to illuminate the anatomical structures ahead of it and/or it may be fitted with a light source of various wave lengths at its tip in order to provide trans-tissue illumination for better distinguishing the anatomical structures of the larynx.

The laryngoscope blade may be fitted with an inbuilt video camera that provides a view onto the anatomical structures ahead of it, which may be displayed on an inbuilt and/or remote monitor screen in the sense of a video-laryngoscope.

The intubation device further comprises at least one guide member, which is substantially rigid and is curved in at least a section thereof.

Preferably, the guide member is curved along its entire length along the advancing direction.

Preferably, the curvature of the guide member is constant along the entire curved section thereof.

In case the laryngoscope blade is curved in at least a section thereof, the curvature of the guide member may be substantially identical or at least similar to the curvature of the curved section of the laryngoscope blade in order to facilitate delivering the endotracheal tube to the patient's trachea.

The intubation device further comprises at least one sled configured to be coupled to the endotracheal tube and advance the endotracheal tube to and/or into the trachea of the patient, and at least one drive device, preferably an electric motor, configured to drive the sled in an advancing direction.

The sled is movably coupled to the guide member such that the sled is translationally movable along the guide member relative to the guide member in the advancing direction.

A translational movement, within the context of the present invention, is to be understood as a movement of a body, in this case the sled, in which all of its points define congruent, either straight or curved, paths.

In the case of a rotational movement of a body, in contrast to a translational movement, all points of the rotating body define coaxial circles, whose planes are oriented perpendicular to an axis of rotation, the centers of which planes lie in the axis of rotation.

Thus, since the sled performs translational movements along the guide member relative to the guide member in the advancing direction, the sled preferably is not pivotally connected to another fixed portion of the intubation device, such as a handle.

The substantially rigid guide member thus may define a travel path along which the sled may be translationally moved in the advancing direction, and/or in a direction opposite thereto, in order to advance and deliver the endotracheal tube to the trachea of the patient. Since the guide member is curved along at least a section thereof, the portion of the travel path along the curved section of the guide member is also curved. This may facilitate delivery of the endotracheal tube to the trachea of the patient.

By providing at least one sled which is movably coupled to the guide member such that the sled may be translationally moved along the guide member relative to the guide member in the advancing direction in order to deliver the endotracheal tube to the trachea of the patient, a particularly compact delivery unit, i.e. the sled, of the endotracheal tube may be provided.

In particular, the sled may at least partially house the drive device, preferably an electric motor, and/or a power source, preferably a battery, for powering the drive device, other drive and/or actuating devices, such as those described below, and/or an engaging member of the sled configured to engage with the guide member to provide the coupling between the sled and the guide member. The coupling may allow the sled to move along and be guided by the guide member. Instead on an electric motor also other drive devices may be considered, such as a hydraulic motor or a pneumatic motor.

The drive device, e.g. the motor, may be configured to advance with the sled.

In contrast, the prior art document US 2017/0304572 A1 mentioned above discloses intubation devices in which a swing arm is configured to rotate about an axis of rotation via an electro-mechanical device or user actuation to deliver an endotracheal tube into a patient's trachea rather than via a translationally movable sled guided along a guide member according to the present invention.

In this case, the swing arm extends from the axis of rotation and protrudes to and past the guide rail, thus occupying a considerable amount of space in the operating environment of the intubation device. Due to the relatively large range of rotational motion of the swing arm and thus the relatively large space the swing arm occupies throughout its rotational motion, the user(s) of the device and/or the patient may collide with the swing arm during operation which may make operation of the device more cumbersome, cause damage to the device and/or even injury to the patient. Thus, the user(s) must be particularly cautious during operation. This reduces the user-friendliness and safety of the device.

In the case of US 2020/0178786 A1, the device disclosed therein does not have a guide member, which is curved at least in a section thereof. Instead, the device has a linear guide to linearly guide the endotracheal tube carriage disclosed therein. The curvature of a primary blade disclosed therein guides and bends the endotracheal tube as the endotracheal tube is being delivered to the patient's trachea. This type of guiding and bending of the endotracheal tube to better conform to the patient's anatomy is less precise and more susceptible to misplacement of the endotracheal tube, which may lead to severe morbidity or even mortality, as discussed at the beginning.

The sled may engage at least a portion of the guide member as the sled is moved translationally in the advancing direction via at least one rotating member, e.g., a gearwheel or a roller. The rotating member is preferably disposed at least partially within the sled.

Additionally, or alternatively, the sled may engage at least a portion of the guide member as the sled is moved translationally in the advancing direction via at least one sliding member, e.g. a sliding bearing and/or a freely rotating member. The sliding member is preferably disposed at least partially within the sled. Moreover, the sliding member may be pressed to the guiding member using springs inside of the sled, i.e. a suspension, wherein the motion of the sliding member my accommodate the changing curvature of the guide member.

The guide member and/or the sled may include cooperating elements, such as grooves and/or protrusions aligned in the advancing direction of the sled, wherein the cooperating elements of the translational sled may cooperate to prevent the sled from rotating or translating in orientations other than the advancing direction or at least reduce such movements.

Other engaging elements via which the sled may engage at least a portion of the guide member as the sled is moved translationally in the advancing direction, such as belts or wires, are also feasible. Moreover, engaging elements relying on friction elements to propel the sled along the guide member may be used, such as soft rubber wheels or hard knurled wheels.

The intubation device may comprise a plurality of guide members, for instance, two or three guide members which are preferably arranged substantially parallel to each other.

Preferably, the drive device is housed within the sled and/or a handle of the device. Alternatively, the drive device may be attached to the guide member.

The drive device may be housed entirely within the sled. This may provide a compact and less obstructive drive device/sled to increase the ergonomics of the intubation device and prevent or at least reduce the risk of collisions between the intubation device and the user's and/or the patient's body.

The drive device may alternatively be housed entirely within the handle of the device.

Alternatively, the drive device may be housed partially within the handle of the device and partially within the sled.

Preferably, the intubation device comprises at least one stylet coupled to the sled. The stylet may comprise a proximal section that is either rigid or flexible. The stylet may comprise at least one bendable section, preferably distal bendable section, configured to be bent in at least one degree of freedom, preferably in at least two degrees of freedom, more preferably in at least three degrees of freedom. Moreover, more degrees of freedom may be utilized to perform maneuvers to ergonomically conform to the anatomy of the patient, e.g. s-bends, sagittal and/or lateral translations, and/or axial rotations.

The stylet may be configured to be positioned at least partially within the endotracheal tube as the endotracheal tube is being delivered to the patient's trachea.

The stylet may be connected to an input interface configured to receive operating commands to bend the stylet at the bendable section.

The stylet may be configured such that the operating commands may be input manually, e.g. via the input interface, by the user to manually manipulate the stylet to bend at the bending section.

The input interface may be provided on a section of the intubation device itself, e.g. on a handle or proximate a handle of the intubation device for easy access of the user. Thus, the input interface may be moved with the intubation device as a single coherent unit.

Alternatively, the input interface may be provided on a remote control. The remote control may be communicatively connectable to the intubation device via a cable or via a wireless connection, such as a Bluetooth connection and/or a telecommunication connection. Such remote control could be provided by a personal computing device (e.g., a smartphone). The personal computing device may be running a dedicated app for this purpose.

Alternatively, or additionally, the stylet may be configured such that the operating commands may be sent automatically by a controller of the intubation device, for instance, based on information related to the anatomy of the patient proximate the pharyngeal cavity. Such information may be provided by a sensor or a plurality of sensors configured to detect information related to the anatomy of the patient. The information may be related to a distance of a portion of the intubation device and/or the endotracheal tube to a portion of the anatomy of the patient proximate the pharyngeal cavity of the patient. As also indicated below, the controller may be integrated into the intubation device, e.g. into a handle of the device, and/or the controller may be provided remotely, e.g. in a remote control connected to the intubation device. As mentioned above, such remote control may be provided by a personal computing device (e.g., a smartphone). The personal computing device may be running a dedicated app for this purpose.

The stylet may be configured to be coupled to the sled such that the stylet is advanced with the sled in the advancing direction towards the patient's trachea.

Preferably, the endotracheal tube is configured to be positioned at least partially over and follow the shape of the stylet and/or the stylet is configured to manipulate at least a section of the endotracheal tube by manipulating at least a section of the stylet, preferably by bending the stylet at the bendable section.

Thus, the stylet may aid in adjusting the position and/or orientation of the endotracheal tube according to the patient's anatomy in order to align the endotracheal tube with the patient's trachea. This may prevent, or at least reduce, the risk of misplacement of the endotracheal tube. Such misplacement may lead to severe morbidity or even mortality of the patient, as detailed above.

Preferably, the intubation device comprises a manipulating mechanism configured to manipulate at least a section of the stylet, preferably by bending the stylet at the bendable section. The manipulating mechanism may comprise at least one angulation wire connected to the manipulating mechanism and extending at least partially through the stylet. The angulation wire may be configured to bend the stylet at the bendable section in at least one degree of freedom.

Preferably, the manipulating mechanism comprises a plurality of angulation wires configured to bend the stylet at the bendable section in multiple degrees of freedom.

The angulation wire(s) may be activated by applying tensile force on the wires, wherein the angulation wire(s) may pull a distal segment of the bendable section causing the bendable section to manipulate about at least one bending axis.

The manipulation mechanism may be driven by manual application of tensile force to the angulation wire(s), wherein the user may manually depress a lever and/or a rotate a handle.

Preferably, the manipulating mechanism may be driven by at least one electric motor coupled to apply tensile force to the angulation wire(s). The manipulating mechanism may be housed in the sled and/or a handle of the device.

The manipulating mechanism may be configured to operate in a manual mode, in which the manipulating mechanism is configured to receive manual user commands and perform manipulation movements, preferably bending at the bendable section, of the stylet based on the manual user commands.

Alternatively or additionally, the manipulating mechanism may be configured to operate in an automatic mode, in which the manipulating mechanism is configured to receive automatic commands from a controller of the intubation device and perform manipulation movements, preferably bending at the bendable section, of the stylet based on the automatic commands.

The manipulating mechanism may be configured to operate in a manual mode and in an automatic mode sequentially or simultaneously.

In the case of operating in the manual mode and in the automatic mode sequentially, the intubation device may have a control element which allows the user to switch the manipulating mechanism from the manual mode to the automatic mode and/or vice versa.

The control may be arranged on the intubation device itself and/or on a remote control.

In the case of operating in the manual mode and in the automatic mode simultaneously, the manipulating mechanism may be configured to allow the user to manipulate the stylet within certain set virtual boundaries, e.g., virtual spatial boundaries, wherein the manipulating mechanism may be configured to override the user's manual operation if the user crosses said virtual boundaries. For instance, the manipulating mechanism may stop manipulation of the stylet. This may help prevent or at least reduce the risk of severe injury to the patient caused by human error while allowing the user some freedom to operate.

The manipulating mechanism may be configured to provide feedback, e.g., haptic and/or visual and/or acoustic feedback, to the user when the virtual boundaries are crossed. For instance, the manipulating mechanism may be configured to vibrate, as a form of haptic feedback, to inform and/or warn the user that the virtual boundaries have been crossed or when the user attempts to cross the virtual boundaries and/or just prior to the virtual boundaries being crosses.

Preferably, the intubation device comprises at least one actuating device, preferably an electric motor, configured to actuate the manipulating mechanism. The actuating device or at least one of the actuating devices is preferably housed in the sled and/or a handle of the device.

Preferably, the intubation device comprises at least one sensor configured to capture the location and/or orientation of at least a section, preferably a distal section or a distal tip, of the laryngoscope blade and/or the endotracheal tube and/or the stylet relative to an anatomy of the patient.

The sensor may be an optical sensor configured to provide optical information of the anatomy of the patient. For instance, such an optical sensor may be a camera sensor or an ultrasound sensor.

The intubation device may be configured to record and store the optical information provided by the optical sensor, e.g., as a video or as images. For this purpose, the intubation device may have an internal memory for storing the optical information and/or the intubation device may have an interface for communicatively connecting to an external memory device, such as a USB stick, for storing the optical information on the external memory device.

The sensor may also be a distance sensor configured to detect a distance to a portion of the anatomy of the patient.

Preferably, the intubation device comprises a plurality of sensors configured to capture the location and/or orientation of at least a section, preferably a distal section or a distal tip, of the laryngoscope blade and/or the stylet relative to an anatomy of the patient.

Preferably, the sensor is an optical sensor, preferably a camera, arranged at a distal tip of the stylet and/or on a portion of the laryngoscope blade. The optical sensor may be configured to provide visual data of an anatomy of the patient.

Preferably, the intubation device further comprises computational circuitry configured to determine if the laryngoscope blade has been correctly positioned. The intubation device may be configured to instruct the user how to correctly place the laryngoscope blade based on visual data provided by the optical sensor.

In other words, the intubation device further comprises computational circuitry configured to determine if the laryngoscope blade has been positioned at a location that is determined by the circuitry to be suitable in relation to the anatomy of the patient. The anatomy of the patient may be identified by the circuitry in the data provided by the optical sensor. The circuitry may be configured to assess whether the position of the intubation device is within a predetermined range in relation to the anatomy identified.

Preferably, the intubation device comprises a controller configured to automatically drive the sled and/or automatically manipulate the stylet. Preferably, the controller is configured to automatically drive the sled and/or automatically manipulate the stylet based on visual data provided by an optical sensor.

For this purpose, the controller may have computational circuitry, such as the computational circuitry discussed above, configured to process data.

The controller may be coupled to at least one sensor configured to capture the location and/or orientation of at least a section, preferably a distal section or a distal tip, of the laryngoscope blade and/or the endotracheal tube and/or the stylet relative to an anatomy of the patient. The sensor may be configured to provide the location and/or orientation of at least a section, preferably a distal section or a distal tip, of the laryngoscope blade and/or the endotracheal tube and/or the stylet relative to an anatomy of the patient to the controller.

Thus, the controller may be configured to automatically drive the sled and/or automatically manipulate the stylet based on the information received from the sensor(s).

The controller may be embedded in the intubation device, for instance in a handle or a housing thereof. Thus, the controller may be moved with the intubation device as a single coherent mobile device.

Instead of having a controller embedded in the intubation device or in addition thereto, data provided by the intubation device, e.g., by any of the sensors provided therein, may be received and processed by an external controller configured to receive and process data from the intubation device and provide command signals to the intubation device, e.g., to automatically drive the sled and/or automatically manipulate the stylet.

The external controller may be communicatively connectable to the intubation device via a cable or wirelessly, for instance, via a Bluetooth connection.

Such an external controller may be a mobile device, such as a smartphone, a tablet, or a smartwatch, or any other computing device, such as a laptop or pc.

Thus, third party devices may be used with the intubation device.

By providing such an external controller, the intubation device may be used more flexibly and intuitively since a common third party device, such as a user's smartphone, may be used as an external controller to communicate with the intubation device.

In any case, the controller may be communicatively connected, by a wire or wirelessly, to the drive device(s) and/or actuating device(s) provided in the intubation device, as described herein, for data transfer and/or power supply purposes.

The external controller may be communicatively connectable to the intubation device via a cable or wirelessly, for instance, via a Bluetooth connection.

The intubation device may also be configured to communicate, either via a wire or wirelessly, with an external data receiving and processing unit.

Preferably, the external data receiving and processing is a mobile computing device, such as a smartphone, a tablet, or a smartwatch, or any other computing device, such as a laptop or pc.

Thus, the intubation device may transfer data, for instance data provided by the sensor(s), to the external data receiving and processing unit, preferably via a transmitting unit arranged on or in the intubation device.

Preferably, the external data receiving and processing unit is configured to process the data and provide information, e.g., visual information, to the user, e.g., via a display of the external data receiving and processing unit.

The external data receiving and processing unit may also provide visual and/or acoustic warnings to the user, e.g., when a misplacement of the endotracheal tube has been detected.

Such an external data receiving and processing unit may be embedded within an external controller, such as the external controller described above. Thus, the external data receiving and processing unit and the external controller may be combined in a single device.

Thus, the intubation device may also be manually controlled via an external device, e.g., a mobile computing device, such as a smartphone, a tablet, or a smartwatch, or any other computing device, such as a laptop or pc.

The intubation device may also be configured to receive data from at least one external data sending device, preferably from a plurality of external data sending devices. This way, the intubation device may be configured to display the data from the external data sending device(s) to the user, e.g., pulse oximetry, blood pressure, and/or temperature of the patient.

Preferably, the stylet has a substantially rigid section. The substantially rigid section may be arranged at a proximal section of the stylet and the bendable section may be arranged at a distal section of the stylet.

Preferably, the intubation device comprises a user interface having at least one manual input device configured to receive manual input commands from a user.

Preferably, the manual input device comprises at least one button and/or at least one switch and/or at least one joystick for receiving manual input commands from a user.

The user interface may be provided on a section of the intubation device itself, e.g., proximate a handle of the intubation device for easy access of the user. Thus, the user interface may be moved with the intubation device as a single coherent mobile unit.

Alternatively, the user interface may be provided on a remote control. The remote control may be communicatively connectable to the intubation device via a cable or via a wireless connection, such as a Bluetooth connection.

Preferably, the user interface comprises at least one display configured to display data provided by the sensor, preferably data provided by the optical sensor.

The user interface may be a virtual user interface provided on an external device, e.g., a mobile computing device, such as a smartphone, a tablet, or a smartwatch, or any other computing device, such as a laptop or pc.

Thus, the virtual user interface may be configured to run on an external device, such as a smartphone, a tablet, or a smartwatch, or any other computing device, such as a laptop or pc, as a graphical user interface.

Preferably, the manual input commands comprise at least one of navigation commands for manually advancing the sled, stylet manipulating commands for manually manipulating at least a section of the stylet, and automation activation commands for activating and/or deactivating an automatic mode of the intubation device in which the sled is driven automatically and/or the stylet is manipulated automatically.

Thus, the automation activation commands for activating and/or deactivating an automatic mode of the intubation device in which the sled is driven automatically and/or the stylet is manipulated automatically may enable the intubation device to be operated in a manual mode and in an automatic mode sequentially by switching from an automated mode to a manual mode and/or vice versa by entering the automation activation commands.

Additionally or alternatively, the intubation device may be configured to be operated in a manual mode and in an automatic mode simultaneously, e.g., by configuring the intubation device to allow the user to operate the intubation device within certain set virtual boundaries, e.g., spatial boundaries, wherein the intubation device may be configured to override the user's manual operation if the user crosses the virtual boundaries. This may help prevent or at least reduce the risk of severe injury to the patient caused by human error while allowing the user at least some freedom to operate.

Preferably, the intubation device comprises at least one illuminating device configured to illuminate the pharyngeal cavity of the patient when the intubation device is in use.

The illuminating device may improve the direct or indirect vision of the user onto the anatomy, in particular the pharyngeal cavity, of the patient. This in turn may improve the ability to assess the location and/or orientation of at least a section, preferably a distal section or a distal tip, of the laryngoscope blade and/or the endotracheal tube and/or the stylet relative to an anatomy of the patient. This may prevent or at least reduce the risk of misplacement of the laryngoscope blade and/or the endotracheal tube and/or the stylet relative to an anatomy of the patient in order to prevent or at least reduce the risk of injury due a portion of the anatomy of the patient.

The illuminating device may be arranged on the blade or on the stylet, preferably at a distal end thereof. The intubation device may comprise a plurality of illuminating devices, with at least one of the plurality of illuminating devices being arranged on the blade and at least one of the plurality of illuminating devices being arranged on the stylet.

Preferably, the intubation device comprises at least one handle configured to be gripped by a hand of a user. The handle may be fixedly connected to at least the guide member and/or the laryngoscope blade. By providing a handle fixedly connected to at least the guide member and/or the laryngoscope blade, a means by which the user can support and/or position the intubation device may be provided.

The handle, or at least a portion thereof, may be arranged substantially in a plane with the sled and/or the guide member and/or the laryngoscope blade. The sled may be configured to move translationally within this plane. This may reduce the intrusiveness and obstructiveness of the intubation device in the operating environment and may increase the compactness, ergonomics and/or user-friendliness of the intubation device. This may prevent or at least reduce the risk of the body of the user or the patient colliding with a portion of the intubation device.

Preferably, the intubation device comprises at least one energy source for powering at least the drive device and/or the actuating device. The energy source(s) may be stored within the sled and/or a separate housing.

The energy source may alternatively be stored at least partially in a handle of the device, preferably a handle as described above.

Preferably, the energy source is at least one rechargeable battery which is preferably rechargeable via an electrical connection port arranged on a portion of the intubation device or via a wireless connection to an electrical source.

Alternatively, the intubation device may be configured for drawing electrical energy directly from the main power supply of a building (such as a hospital) or a vehicle electrical system (such as an on-board system of an ambulance or airplane). The intubation device may be provided with a cable and plug for connecting to the electrical system. A battery may not be required.

Preferably, the guide member is a toothed rack. Preferably, said toothed rack is curved in at least a section thereof.

Preferably, the sled comprises a gearwheel driven by the drive device, wherein the gearwheel is configured to engage at least a portion of the guide member to translationally move the sled in the advancing direction.

The sled may alternatively comprise other elements, such as sliders, gears, belts, or wires. Such elements may be configured to engage at least a portion of the guide member to translationally move the sled in the advancing direction along a portion of the guide member.

For example, according to an alternative configuration, the guide member may be a curved rail and the sled may comprise one or more driven rollers (e.g., rubber rollers) rolling along said curved rail to advance the sled. In this context, the rail may be clamped between one or more pairs of rollers of the sled. As the skilled person will appreciate, one driven roller may be sufficient for advancing the sled, but also several driven rollers could be provided, e.g. one per pair of rollers clamping the rail.

According to a further alternative configuration, the sled may be advanced and/or retracted via belt or wire. For example, the guide member may be a rail and the sled may be advanced by a belt or wire extend along the rail. The sled could be fixed to said belt or wire. The wire could then be moved in order to advance and/or retract the sled along the guide member. The belt or wire may be moved by a drive device (e.g., a motor) that is attached to the guide member and/or separate from the sled.

According to a further alternative configuration, the sled may be advanced and/or retracted via a spindle drive. For example, the guide member may be a rail and the sled may be advanced by a separate spindle, in particular a flexible spindle extending substantially parallel to the rail. In this case, the sled may comprise a spindle nut that moves along the spindle. The spindle may be driven by a drive device (e.g., a motor) that is attached to the guide member and/or separate from the sled.

Preferably, the sled and/or the guide member and/or a handle of the device comprise a pulley wheel driven by the drive device. Preferably, the pulley wheel is configured to engage a cable anchored in the guide member and/or the sled and/or the handle. The cable may be oriented by a plurality of idler wheels.

Preferably, the sled comprises a quick release coupling mechanism for coupling and releasing the endotracheal tube to and from the sled. The sled may also incorporate a quick release coupling mechanism for coupling and releasing the sled to the guide member, such that the sled can be physically removed from the guide member, e.g., for sanitization and disinfection of certain components of the device.

The quick release coupling mechanism for the endotracheal tube may be configured as a bayonet coupling. The quick release coupling mechanism may alternatively be configured as a luer lock or a luer slip coupling.

The quick release coupling mechanism may also be configured as an interference fit coupling.

Alternatively, the quick release coupling mechanism may comprise at least one protrusion provided on one of the endotracheal tube and the sled and at least one opening configured to receive the protrusion, the opening being provided on the other of the endotracheal tube and the sled.

By receiving the protrusion in the opening the endotracheal tube may be temporarily fixed to the sled. The endotracheal tube and/or the sled may be configured such the protrusion can be released from the opening when it is desired to disconnect the endotracheal tube from the sled, such as when the tracheal intubation process has been completed.

Preferably, the laryngoscope blade is curved in at least a section thereof.

Preferably, the intubation device comprises a remote controller configured to drive the sled and/or manipulate the stylet. The controller may be configured to drive the sled and/or manipulate the stylet based on a remote user providing navigation commands based on visual data provided by an optical sensor.

The object mentioned at the beginning is also solved by an intubation device for providing assistance to a user in delivering an endotracheal tube to a trachea of a patient according to a further aspect of the present disclosure, as defined by the features of independent claim 11. Variations and further developments are defined by the features of the respective dependent claims. The embodiments and/or features and/or advantages described above with respect to the intubation device according to the first aspect of the present disclosure apply to the intubation device according to the further aspect accordingly.

The intubation device comprises at least one laryngoscope blade for providing access to a pharyngeal cavity of the patient. The laryngoscope blade may be curved in at least a section thereof. This may facilitate the delivery of the endotracheal tube to the patient's trachea. The curvature of the laryngoscope blade may be determined based on the anatomy of the patient, for instance, based on size and/or age and/or gender of the patient. Thus, a variety of laryngoscope blades having different curvatures and/or other different geometric properties may be provided in order to adapt the shape and/or size of the laryngoscope blade to different patients having different anatomies.

Different laryngoscope blades may be attached to and released from the intubation device, e.g., from a handle of the device, in a modular manner, for instance, via a coupling mechanism, preferably a quick release coupling mechanism, such as a bayonet coupling.

The laryngoscope blade may define a channel through at least a section of the laryngoscope blade, the channel being configured to at least partially receive the endotracheal tube.

The laryngoscope blade may be fitted with a light source to illuminate the anatomical structures ahead of it and/or it may be fitted with a light source of various wave lengths at its distal tip in order to provide trans-tissue illumination for better distinguishing the anatomical structures of the larynx.

The intubation device further comprises at least one sensor configured to capture the location and/or orientation of at least a section, preferably a distal section or a distal tip, of the laryngoscope blade and/or the endotracheal tube relative to an anatomy of the patient. Thus, the intubation device, preferably the laryngoscope blade, may be provided with one or more sensors mounted thereon, preferably one or more video sensors, that are configured to capture data related to the location and/or orientation of at least a section, preferably a distal section or a distal tip, of the laryngoscope blade and/or the endotracheal tube relative to an anatomy of the patient, e.g., relative to one or more features of one or more anatomical structures of the patient, which preferably lie ahead of the laryngoscope blade and/or the endotracheal tube in a direction of insertion of the laryngoscope blade and/or the endotracheal tube into the patient, during operation of the intubation device.

The data may be provided to the user, e.g., visually and/or acoustically and/or haptically.

Preferably, the sensor may be configured to capture one or more images of the laryngoscope blade and/or the endotracheal tube and/or an anatomy of the patient. The intubation device may be configured to visualize said one or more images to the user, e.g., by means of one or more screens, which may be included and/or integrated in the intubation device as a component thereof. Alternatively, one or more external screens, e.g., remote screens, may be communicatively couplable to the intubation device, more specifically to the sensor, to provide said one or more images to the user.

Preferably, the sensor is an optical sensor, preferably a video sensor, which is preferably mounted on a distal tip or a distal section of the laryngoscope blade. The optical sensor may be configured to provide visual data, preferably live visual data, preferably continuous visual data at least over a certain time span, of the laryngoscope blade and/or the endotracheal tube and/or an anatomy of the patient during operation of the intubation device. However, the sensor may be any sensor configured to capture and/or sense information related to the location and/or orientation of at least a section, preferably a distal section or a distal tip, of the laryngoscope blade and/or the endotracheal tube relative to an anatomy of the patient. For instance, the sensor may be configured as a tactile sensor and/or an ultrasonic sensor.

The intubation device further comprises computational circuitry configured to determine if the laryngoscope blade has been correctly positioned, wherein the intubation device is configured to instruct the user how to correctly place the laryngoscope blade based on data, preferably visual data, provided by the sensor.

By configuring the intubation device to instruct the user how to correctly place the laryngoscope blade based on data, preferably visual data, provided by the sensor, the intubation device described herein may provide instructional guidance to the user for placing the laryngoscope blade in the patient's pharyngeal cavity during operation of the intubation device, e.g., as the laryngoscope blade is being inserted into the patient's pharyngeal cavity, to allow the endotracheal tube to be inserted into the patient's trachea. This may reduce the risk of misplacement of the intubation device, e.g., the laryngoscope blade, e.g., in the pharyngeal cavity of the patient, e.g., due to human error and/or difficult and stressful circumstances and/or anatomical factors and/or systemic conditions. As a result, the intubation device described herein may be less prone to errors, particularly human errors, than laryngoscopes, including video-laryngoscopes, which are known from the prior art.

The computational circuitry may be configured to receive data, e.g., one or more input signals related to the information captured by the sensor, e.g., the location and/or orientation of at least a section, preferably a distal section or a distal tip, of the laryngoscope blade and/or the endotracheal tube relative to an anatomy of the patient, and process and/or analyze said data. The data may include one or more images, a live video feed and/or a recorded video captured by the sensor.

The computational circuitry may be configured to process the data and at least determine a position and/or an orientation of the laryngoscope blade, preferably at least a distal end thereof, relative to the anatomy of the patient.

Preferably, in order to determine if the laryngoscope blade has been correctly positioned, the computational circuitry may be configured to determine if the laryngoscope blade has reached a target position, preferably a predetermined target position, relative to the patient's anatomy and/or if the laryngoscope blade is within a certain, preferably predetermined, range of the target position. Alternatively, or additionally, the computational circuitry may be configured to determine if the laryngoscope blade has reached a target orientation, preferably a predetermined target orientation, relative to the patient's anatomy and/or if the laryngoscope blade is within a certain, preferably predetermined, range of the target orientation.

Alternatively, or additionally, the computational circuitry may be configured to determine at least one deviation between a current position and/or a current orientation of the laryngoscope blade relative to the patient's anatomy and a target position, preferably a predetermined target position, and a target orientation, preferably a predetermined target orientation, respectively, relative to the patient's anatomy. The computational circuitry may be configured to provide one or more warning signals, if the deviation exceeds a certain, preferably predetermined, threshold and/or if a distance between a section of the laryngoscope blade and the patient's anatomy is lower than a certain, preferably predetermined, threshold.

Alternatively, or additionally, the computational circuitry may be configured to determine one or more maneuvers of the intubation device and/or of the laryngoscope blade, e.g., one or more translational movements and/or one or more rotational movements, which the user may perform in order to move the intubation device and/or the laryngoscope blade to, or at least towards, a target position, preferably a predetermined target position, and/or to, or at least towards, a target orientation, preferably a predetermined target orientation, respectively, of the laryngoscope blade relative to the patient's anatomy.

The determinations and/or instructions described above may be provided to the user, e.g., visually, such as on a display screen and/or in a graphical user interface, acoustically and/or by means of tactile and/or haptic feedback.

The computational circuitry may be configured to process and/or analyze data from one or more sensor sources, e.g., including a raw video feed from at least one video sensor and/or one or more auxiliary sensors, such as one or more inertial measuring devices, e.g., one or more inertial measurement units (IMU), and/or one or more load measuring devices, such as one of more load cells, e.g., to determine a force which is exerted against an anatomy of the patient, e.g., the patient's tongue, by the user via the intubation device and/or the laryngoscope blade. For instance, the computational circuitry may be configured to process and/or analyze a raw video feed, e.g., provided by one or video sensors, and further (supplementary) data provided by one or more auxiliary sensors, e.g., measurement data related to a trans-tissue illumination of an anatomy of the patient provided by one or more light sources, an ultrasonic tissue transmission and/or imaging of the patient's anatomy provided by one or more x-ray sources and/or radio frequency sources.

The computational circuitry may be integrated and/or embedded in at least one component of the intubation device, e.g., in a controller of the intubation device, e.g., a controller which is configured to control a stylet which is configured to manipulate and/or maneuver the endotracheal tube.

Alternatively, or additionally, the computational circuitry may be provided by an external device, such as a personal computing device, e.g., a smartphone, a tablet, a smartwatch, a laptop, a pc or any other computing device, which is communicatively couplable, e.g., via a cable and/or a wireless connection, such as a Bluetooth connection and/or a telecommunication connection, to the intubation device, e.g., the sensor. Alternatively, or additionally, the computational circuitry may be provided by one or more cloud computing servers with which the intubation device may be communicatively couplable.

Preferably, the intubation device comprises at least one stylet which includes at least one bendable section configured to be bent in at least one degree of freedom, preferably at least two degrees of freedom, more preferably at least three degrees of freedom. Preferably, the endotracheal tube is configured to be positioned at least partially over and follow the shape of the stylet. Alternatively, or additionally, the stylet is configured to manipulate at least a section of the endotracheal tube by manipulating at least a section of the stylet, preferably by bending the stylet at the bendable section.

Preferably, the intubation device comprises a manipulating mechanism configured to manipulate at least a section of the stylet, preferably by bending the stylet at the bendable section. The manipulating mechanism preferably comprises at least one angulation wire connected to the manipulating mechanism and extending at least partially through the stylet. Preferably, the angulation wire is configured to bend the stylet at the bendable section in at least one degree of freedom.

Alternatively, or additionally, the intubation device may comprise a controller configured to automatically manipulate the stylet, preferably based on visual data provided by the sensor.

The invention also relates to a kit comprising an intubation device according to any of the embodiments described herein and an external data and processing receiving unit communicatively connectable to the intubation device and configured to receive and process data received by the intubation device and/or send data to the intubation device.

Thus, instead of having an embedded controller and computational circuitry configured to receive and process data of the intubation device, such as data provided by any of the sensors provided therein, an external data receiving and processing unit configured to receive and process data received by the intubation device may be provided.

The external data receiving and processing unit may be a mobile device, such as a smartphone, a tablet, or a smartwatch, or any other computing device, such as a laptop or pc.

Thus, third party devices may be used with the intubation device.

The external data receiving and processing unit may be communicatively connectable to the intubation device via a cable or wirelessly, for instance, via a Bluetooth connection.

By providing such external data receiving and processing unit, the intubation device can be used more flexibly and intuitively since a common third-party device, such as a user's smartphone, can be used as an external data receiving and processing unit to communicate with the intubation device.

The following list of aspects provides alternative and/or further features of the invention:
1. An intubation device for delivering an endotracheal tube to a trachea of a patient, the intubation device comprising:
   at least one guide member which is substantially rigid and is curved in at least a section thereof;
   at least one advancing member, preferably a sled, configured to be coupled to the endotracheal tube and advance the endotracheal tube to the trachea of the patient;
   at least one drive device, preferably an electric motor, configured to drive the advancing member in an advancing direction; and
   preferably also at least one laryngoscope blade for providing access to a pharyngeal cavity of the patient;
   wherein the advancing member is movably coupled to the guide member such that the advancing member is translationally movable along the guide member relative to the guide member in the advancing direction.
2. The intubation device according to aspect 1, wherein the drive device is housed within the advancing member and/or a handle of the device.
3. The intubation device according to aspect 1 or 2, comprising at least one stylet coupled to the advancing member, wherein the stylet comprises at least one bendable section configured to be bent in at least one degree of freedom, preferably at least two degrees of freedom, more preferably at least three degrees of freedom.
4. The intubation device according to aspect 3, wherein the endotracheal tube is configured to be positioned at least partially over, and preferably, follow the shape of the stylet; and/or
   wherein the stylet is configured to manipulate at least a section of the endotracheal tube by manipulating at least a section of the stylet, preferably by bending the stylet at the bendable section.
5. The intubation device according to aspect 3 or 4, comprising a manipulating mechanism configured to manipulate at least a section of the stylet, preferably by bending the stylet at the bendable section.
6. The intubation device according to aspect 5, comprising at least one actuating device, preferably an electric motor, configured to actuate the manipulating mechanism, wherein the actuating device is preferably housed in the advancing member and/or in a handle of the device.
7. The intubation device according to aspect 6, wherein the manipulating mechanism comprises at least one manipulating wire, preferably an angulation wire, connected to the actuating device and/or to the manipulating mechanism and extending at least partially through the stylet, wherein the manipulating wire is configured to bend the stylet at the bendable section in at least one degree of freedom.
8. The intubation device according to any of the preceding aspects, comprising at least one sensor configured to capture the location and/or orientation of at least a section, preferably a distal section or a distal tip, of the laryngoscope blade and/or the endotracheal tube and/or the stylet relative to an anatomy of the patient.
9. The intubation device according to aspect 8, wherein the sensor is an optical sensor, preferably a camera, arranged at a distal tip of the stylet and/or on a portion of the laryngoscope blade, the optical sensor being configured to provide visual data of an anatomy of the patient.
10. The intubation device according to aspect 9, further comprising computational circuitry configured to determine if a part of the intubation device, preferably the laryngoscope blade, has been correctly positioned, wherein the intubation device is configured to instruct the user how to correctly place the part, preferably the laryngoscope blade, preferably based on visual data provided by the optical sensor.
11. The intubation device according to any of the preceding aspects, comprising a controller configured to automatically drive the advancing member and/or automatically manipulate the stylet.
12. The intubation device according to aspect 11 when dependent on aspect 9 or 10, wherein the controller is:
   configured to automatically, or at least semi-automatically, drive the advancing member and/or automatically manipulate the stylet based on the visual data provided by the optical sensor; and/or
   configured to be driven remotely; and/or
   configured to give acoustic and/or visual instructions.
13. The intubation device according to any of aspects 3 to 12, wherein the stylet has a substantially rigid section, wherein preferably the substantially rigid section is arranged at a proximal section of the stylet and the bendable section is arranged at a distal section of the stylet.
14. The intubation device according to any of the preceding aspects, comprising a user interface having at least one manual input device configured to receive manual input commands from a user.
15. The intubation device according to aspect 14 when dependent on aspect 8 or 9, wherein the user interface comprises at least one display configured to display data provided by the sensor, preferably data provided by the optical sensor.
16. The intubation device according to aspect 14 or 15, wherein the manual input commands comprise at least one of navigation commands for manually advancing the advancing member, stylet manipulating commands for manually manipulating at least a section of the stylet, and automation activation commands for activating and/or deactivating an automatic mode of the intubation device in which the advancing member is driven automatically and/or the stylet is manipulated automatically.
17. The intubation device according to any of aspects 14 to 16, wherein the manual input device comprises at least one button and/or at least one switch and/or at least one joystick for receiving manual input commands from a user.
18. The intubation device according to any of the preceding aspects, comprising at least one illuminating device configured to illuminate an anatomy of the patient, preferably at least the pharyngeal cavity of the patient, when the intubation device is in use.
19. The intubation device according to any of the preceding aspects, comprising at least one handle configured to be gripped by a hand of a user, wherein the handle is fixedly connected to at least the guide member and/or the laryngoscope blade.
20. The intubation device according to any of the preceding aspects, comprising an energy source for powering at least the drive device and/or the actuating device, the energy source being stored within the advancing member and/or a separate housing.
21. The intubation device according to aspect 20, wherein the energy source is at least one rechargeable battery which is preferably rechargeable via an electrical connection port arranged on a portion of the intubation device or via a wireless connection to an electrical source.
22. The intubation device according to any of the preceding aspects, wherein the guide member is a toothed rack which is curved in at least a section thereof.
23. The intubation device according to any of the preceding aspects, wherein the advancing member comprises a gearwheel driven by the drive device, wherein the gearwheel is configured to engage at least a portion of the guide member to translationally move the advancing member in the advancing direction.
24. The intubation device according to any of the preceding aspects, wherein the sled and/or the guide member and/or a handle of the device comprise a wheel, preferably a pulley wheel, driven by the drive device, wherein the wheel is preferably configured to engage a cable anchored on the guide member and/or the sled and/or the handle, wherein the cable may be oriented by a plurality of idler wheels.
25. The intubation device according to any of the preceding aspects, wherein the advancing member comprises a quick release coupling mechanism for coupling and releasing the endotracheal tube to and from the advancing member.
26. The intubation device according to any of the preceding aspects, comprising a remote controller configured to drive the sled and/or manipulate the stylet, wherein the controller is preferably configured to drive the sled and/or manipulate the stylet, preferably based on a remote user providing navigation commands, preferably based on visual data provided by an optical sensor.
27. An intubation device according to any of the preceding aspects, wherein the translational sled assembly is physically detachable from the guide member.
28. An intubation device for providing assistance to a user in delivering an endotracheal tube to a trachea of a patient, the intubation device comprising:
   at least one laryngoscope blade for providing access to a pharyngeal cavity of the patient;
   at least one sensor configured to capture the location and/or orientation of at least a section, preferably a distal section or a distal tip, of the laryngoscope blade and/or the endotracheal tube relative to an anatomy of the patient; and
   at least one computing device, preferably at least one computational circuitry, configured to determine at least a position and/or an orientation of the laryngoscope blade, preferably at least a distal end thereof, relative to the anatomy of the patient and/or if the laryngoscope blade has been correctly positioned, wherein the intubation device is configured to instruct the user how to correctly place the laryngoscope blade and/or how to reach a target position and/or a target orientation of the laryngoscope blade relative to the patient's anatomy based on data, preferably visual data, provided by the sensor.
29. The intubation device according to aspect 28, wherein the computational circuitry is configured to determine at least one maneuver of the intubation device and/or of the laryngoscope blade to move the intubation device and/or the laryngoscope blade to, or at least towards, a target position, preferably a predetermined target position, and/or to, or at least towards, a target orientation, preferably a predetermined target orientation, respectively, of the laryngoscope blade relative to the patient's anatomy based on data, preferably visual data, provided by the sensor.
30. The intubation device according to aspect 28 or 29, comprising at least one stylet which includes at least one bendable section configured to be bent in at least one degree of freedom, preferably at least two degrees of freedom, more preferably at least three degrees of freedom;
   preferably wherein:
   the endotracheal tube is configured to be positioned at least partially over and follow the shape of the stylet; and/or
   the stylet is configured to manipulate at least a section of the endotracheal tube by manipulating at least a section of the stylet, preferably by bending the stylet at the bendable section.
31. The intubation device according to aspect 30, comprising:
   a manipulating mechanism configured to manipulate at least a section of the stylet, preferably by bending the stylet at the bendable section, wherein the manipulating mechanism comprises at least one angulation wire connected to the manipulating mechanism and extending at least partially through the stylet, wherein the angulation wire is configured to bend the stylet at the bendable section in at least one degree of freedom; and/or
   a controller configured to automatically manipulate the stylet, preferably based on visual data provided by the sensor.
32. The intubation device according to any of aspects 28 to 31, wherein the sensor is an optical sensor, preferably a camera, arranged at a distal tip of the stylet and/or on a portion of the laryngoscope blade, the optical sensor being configured to provide visual data of the anatomy of the patient.
33. The intubation device according to any of aspects 28 to 31, further comprising the features according to any of aspects 1 to 27.
34. A kit comprising an intubation device according to any of the preceding aspects and an external data receiving and processing unit communicatively connectable to the intubation device and configured to receive and process data received by the intubation device.

Preferred embodiments of the present invention are further elucidated below with reference to the figures. The described embodiments do not limit the present invention.

### BRIEF DESCRIPTION OF THE DRAWINGS

FIG. 1 shows a perspective view of an intubation device according to an embodiment of the invention with the sled in a retracted position;

FIG. 2 shows the intubation device according to FIG. 1 with an endotracheal tube attached to the sled, and the sled in an advanced position;

FIG. 3 shows the intubation device according to FIG. 1 with the endotracheal tube attached to the sled, and the sled in an advanced position;

FIG. 4 shows the intubation device with the endotracheal tube attached to the sled according to FIG. 3;

FIG. 5 shows the intubation device with the endotracheal tube attached to the sled according to FIGS. 3 and 4;

FIG. 6 shows the intubation device according to FIG. 2 with an alternative pulley style drive device mechanism according to another embodiment of the invention;

FIG. 7 shows a process flowchart for actions and determinations of the intubation device from intubation setup to device navigation to intubation;

FIG. 8 shows a graphical user interface and a mode of operation of the intubation device.

### DETAILED DESCRIPTION OF THE DRAWINGS

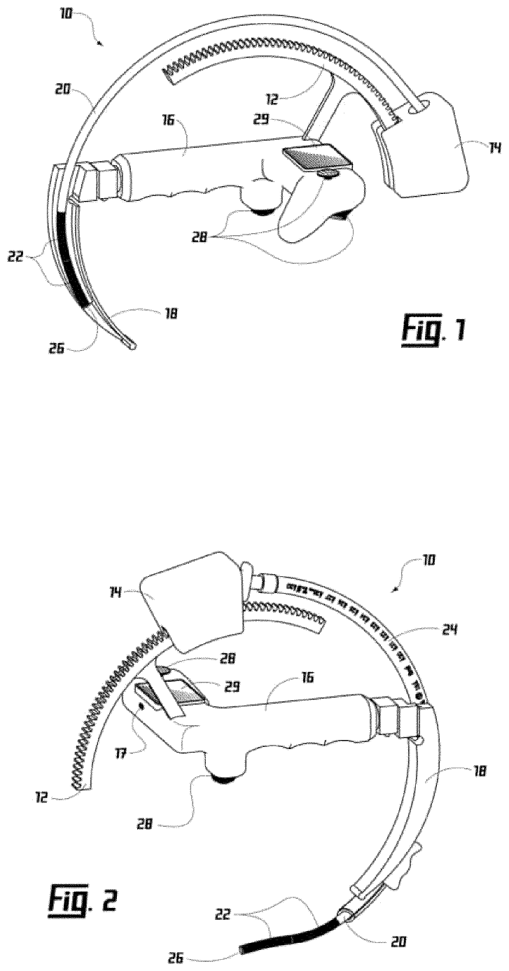

FIGS. 1 to 5 show perspective views of an intubation device 10 for delivering an endotracheal tube to a trachea of a patient according to a first embodiment of the invention. FIG. 6 shows a perspective view of a second embodiment of the invention, which is similar to the first embodiment shown in FIGS. 1 to 5. The main differences between the first and the second embodiment will be discussed further below.

The intubation device 10 comprises a curved and substantially rigid guide member 12. As shown in FIGS. 1 to 6, the guide member 12 is curved along its entire length. However, the guide member 12 may also be curved in only a section thereof. Thus, a section of the guide member 12 may not have a curvature, i.e., the guide member may be substantially straight in such a section.

In the embodiment shown in FIGS. 1 to 5 the guide member 12 is configured as a toothed rack having a plurality of teeth as an engaging means. Alternatively, the guide member 12 may be configured as any other element, which may provide engaging means.

Thus, the guide member 12 is not necessarily required to have teeth. Instead, the guide member 12 may, for instance, have a substantially uniform outer surface as an engaging surface instead of the teeth shown in FIGS. 1 to 5 and the sled 14 may have at least one slider or roller which slides or rolls, respectively, along the outer surface of the guide member 12 to translationally move the sled 14 in the advancing direction and/or in a direction opposite to the advancing direction along at least a portion of the guide member 12.

An alternatively configured guide member 12 with alternatively configured engaging means is shown in the embodiment shown in FIG. 6, which is discussed further below.

The intubation device 10 further comprises a sled 14 configured to be coupled to an endotracheal tube and advance the endotracheal tube to the patient's trachea.

The intubation device 10 also comprises a drive device 30 (not shown in FIGS. 1 to 5), preferably an electric motor, configured to drive the sled 14 in an advancing direction, and preferably also in a direction opposite to the advancing direction. The drive device is preferably housed within the sled 14.

The sled 14 is movably coupled to the guide member 12 such that the sled 14 is translationally movable along the guide member 12 relative to the guide member 12 in the advancing direction.

The sled 14 may be coupled to the guide member 12 via a rotatably mounted gearwheel (not shown) having a plurality of teeth which may engage the teeth on the toothed rack of the guide member 12.

The gearwheel may be rotatably driven by the drive device such that the gearwheel is rotated and moved along the toothed rack in the advancing direction and/or in a direction opposite to the advancing direction.

The intubation device 10 also comprises a handle 16 configured to be gripped by a hand of a user. The handle 16 is fixedly connected to the guide member 12. In particular, the handle 16 may be fixedly attached to the guide member 12 by one or more connectors, e.g. by a single connector as shown in FIG. 1.

The handle 16 may be configured as a housing, which may house components of the intubation device 10, such as the drive device described above, further drive and/or actuating devices, a controller, computational circuitry and/or one or more power sources for powering the intubation device 10, preferably one or more rechargeable batteries. Thus, the handle 16 may provide protection to said components against dust and/or liquids.

The intubation device 10 also comprises an electrical connection port 17 by means of which a rechargeable internal power source, e.g., at least one rechargeable battery, arranged in a section of the intubation device 10 may be charged by an external power source.

Alternatively, such a rechargeable internal power source may be recharged by means of wireless charging, e.g., inductively.

The power source may alternatively be an external supply of power connected to the intubation device 10, e.g., via an electrical cable (not shown).

According to the embodiment shown in FIGS. 1 to 6, the handle 16 is configured to be ergonomic in the left hand of the operator. This may allow the user to be positioned at the head of the patient, facing the patient, as the patient is lying on his/her back during the intubation procedure. The user can thereby grip the intubation device 10 at the handle 16 with his/her left hand while operating the intubation device 10 and performing the tracheal intubation procedure.

Depending on the dexterity type of the user, different handles 16 with different configurations, i.e., handles with higher ergonomics for left-handed or right-handed users, may be provided.

A handle 16 may be provided which may be adjusted and adapted to the dexterity type of the user.

The intubation device 10 further comprises a curved laryngoscope blade 18 for providing access to a pharyngeal cavity of the patient, e.g. by depressing the tongue of the patient and opening the pharyngeal cavity of the patient. The laryngoscope blade 18 is fixedly connected to the handle 16.

The intubation device 10 further comprises a stylet 20 coupled to the sled 14. The stylet 20 comprises at least one bendable section 22 configured to be bent in at least one degree of freedom, preferably at least two degrees of freedom, more preferably at least three degrees of freedom.

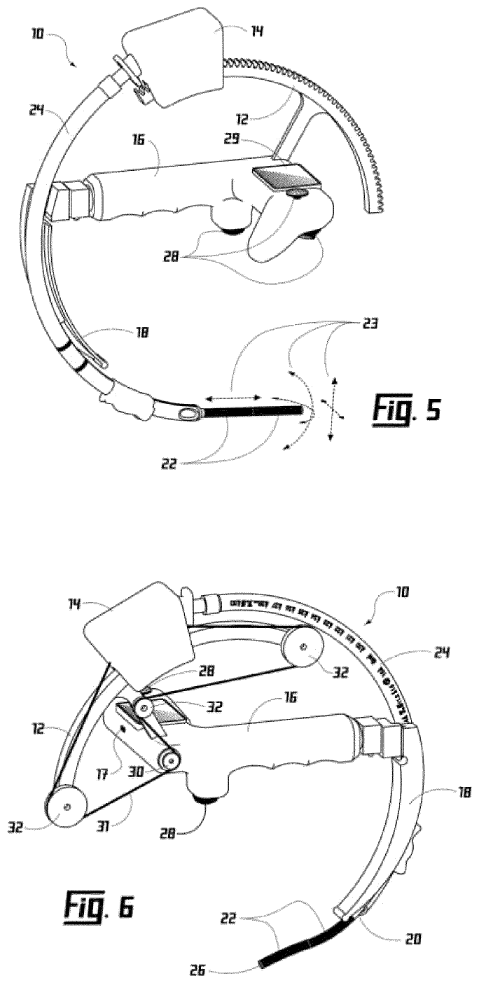

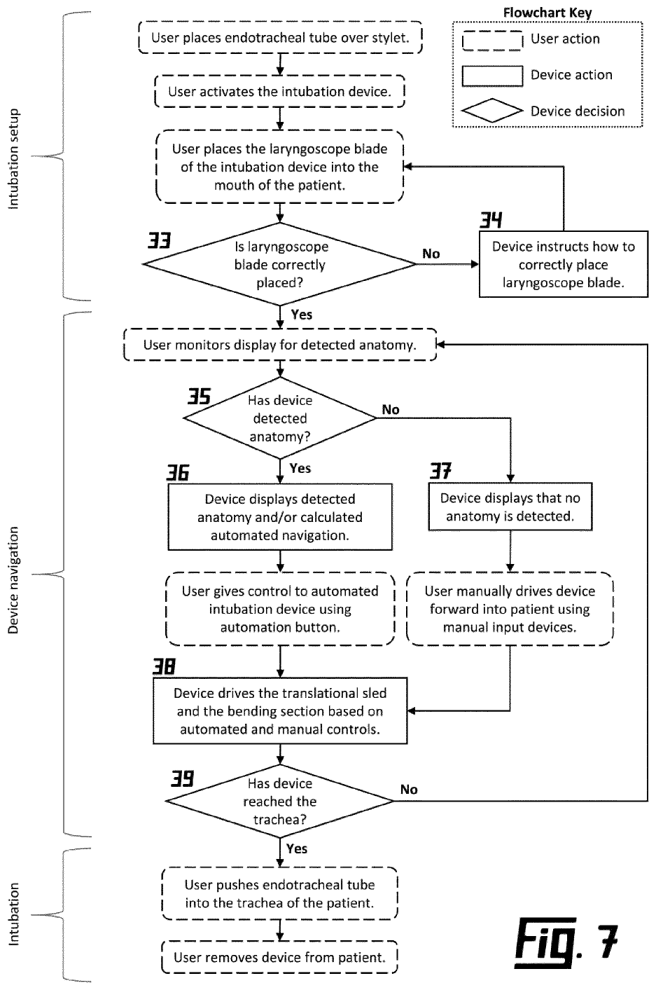

According to the embodiment shown in FIGS. 1 to 6, the stylet 20 is configured to be maneuvered in multiple degrees of freedom 23, i.e. by moving a distal section of the stylet 20 in directions along a longitudinal axis of the distal section, by moving the distal section of the stylet 20 in directions transverse to the longitudinal axis and/or by bending a section of the stylet 20 about axes which are transverse to the longitudinal axis (see FIG. 5).

Thus, as shown in FIGS. 1 to 6, the stylet 20 may comprise at least one bendable section, preferably 2 bendable sections 22, wherein the bendable sections 22 may simultaneously be bent in different, e.g., opposite, directions.

The bendable sections may be bent via a manipulating mechanism (not shown) configured to manipulate at least a section of the stylet 20, preferably by bending the stylet at the bendable sections 22.

The manipulating mechanism may comprise an actuating device, preferably an electric motor, configured to actuate the manipulating mechanism, preferably having at least one angulation wire connected to the actuating device and extending at least partially through or along the stylet 20. The one or more angulation wires may run to the sled 14

The manipulating mechanism, e.g., the angulation wire(s), may be configured to bend the stylet 20 at the bendable section(s) 22. The actuating device may be housed at least partially in the sled 14 and/or in the handle 16.

Alternatively or additionally, bending of one or more bendable sections 22 may be achieved by the stylet including one or more bending elements (not shown) made from a shape memory alloy which may be heated to deform the stylet. For example, one or more shape memory alloy wires may be provided along the stylet, which may be heated to achieve bending of a bendable section 22. The one or more bending elements may be supplied with power via the sled 14.

Sections of the stylet 20 other than the bendable sections 22 may be substantially rigid, semi-rigid, or flexible.

The bending sections 22 of the stylet 20 may have various configurations for achieving a desired maximum bending angle and a desired minimum bending radius of the respective bending section 22.

An endotracheal tube 24 may be positioned at least partially over the stylet 20 such that the endotracheal tube 24 follows the shape of the stylet 20 (see FIGS. 2 to 5).

The stylet 20 may be configured to manipulate at least a section of the endotracheal tube 24 by means of the manipulating mechanism mentioned above.

Thus, by manipulating the stylet 20, e.g., by bending the stylet 20 at at least one bendable section 22 via the manipulating mechanism and the angulation wire(s), the stylet 20 may transfer a force to at least a portion of the endotracheal tube 24 such that the endotracheal tube 24 follows the stylet 20 at least partially.

Thus, the stylet 20 may adjust the positioning and/or orientation of at least a section of the endotracheal tube 24, e.g., according to the anatomy of the patient, in order to facilitate delivery of the endotracheal tube 24 into the patient's trachea.

For instance, the stylet 20 may adjust the curvature of the endotracheal tube 24.

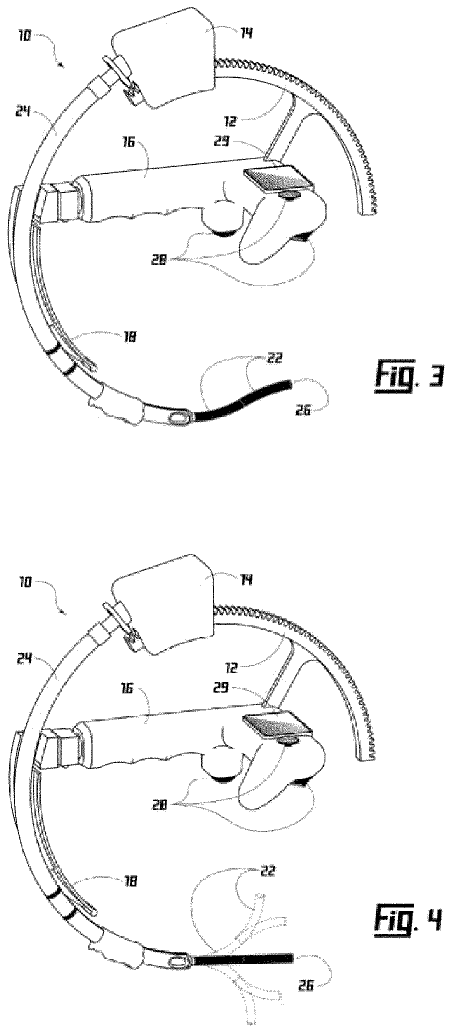

The manipulation of the stylet 20 at its bendable sections 22 is best seen in FIG. 4, which shows different positions and orientations into which the stylet 20 can be brought via the manipulating mechanism in order to thereby adjust the positioning and/or orientation of at least a section of the endotracheal tube 24.

The intubation device 10 further comprises an optical sensor 26 arranged at a distal tip of the stylet 20. The optical sensor 26 may be configured to capture the location and/or orientation of at least a section, preferably a distal section or a distal tip, of the laryngoscope blade 18 and/or the endotracheal tube 24 and/or the stylet 20 relative to an anatomy within and/or proximate the pharyngeal cavity of the patient and provide visual data of said anatomy.

The optical sensor 26 may thus aid in guiding the stylet 20, and thus the endotracheal tube 24, into the patient's trachea by providing visual data of the patient's anatomy.

Thus, for instance, the intubation device 10 may comprise a controller configured to automatically drive the sled 14 in the advancing direction and/or automatically manipulate the stylet 20 based on the visual data provided by the optical sensor 26.

The controller may be embedded in the intubation device or may alternatively be an external controller communicatively connectable to the intubation device 10.

The optical sensor 26 may be a fiberoptic or any digital image sensor including, but not limited to, CMOS and CCD.

The intubation device 10 may comprise a plurality of optical sensors 26. The plurality of optical sensors 26 may be arranged at different locations of the intubation device 10.

For instance, the intubation device 10 may have one optical sensor 26 arranged at a distal tip of the stylet 20, as shown in FIGS. 1 to 6, and one optical sensor (not shown) arranged on a section of the laryngoscope blade 18. The intubation device 10 may also have a plurality of optical sensors 26 arranged on the stylet 20, each of them preferably arranged at or proximate the distal tip thereof.

The intubation device 10 may further comprise any series of sensors for determining the state of the device and/or the intubation procedure. For example, the sled 14 and/or the handle 16 may comprise inertial sensors and/or thermal sensors. Alternatively or additionally, the stylet 20 and/or the laryngoscope blade 18 may comprise load sensors.

The intubation device 10 further comprises a user interface which includes one or more manual input devices 28 configured to receive manual input commands from the user. The one or more manual input devices 28 may be configured as buttons, switches and/or joysticks. Each manual input device 28 may be configured differently, e.g., one manual input device 28 may be configured as a button while another manual input device 28 may be configured as a joystick.

In a preferred embodiment, the user interface may comprise a button or switch for activating the power of the intubation device 10, a button or switch for activating the automatic mode of the intubation device 10, one or more thumbsticks or joysticks for commanding the position and/or orientation of tip of the stylet 20 (and/or of the optical sensor 26 via the stylet 20).

FIGS. 1 to 6 show three manual input devices 28. However, the intubation device 10 may have any number of manual input devices 28. The intubation device 10 may also only have one manual input device 28.

The manual input device 28 may also be configured as a joystick on which a button is provided which is configured to be pushed in order to receive manual input commands by the user.

The manual input commands may comprise navigation commands for manually advancing the sled 14 in the advancing direction and/or in a direction opposite to the advancing direction, stylet manipulating commands for manually manipulating at least a section of the stylet 20, and/or automation activation commands for activating and/or deactivating an automatic mode of the intubation device 10 in which the sled 14 is driven automatically in the advancing direction and/or the stylet 20 is manipulated automatically.

The intubation device 10 further comprises a display 29 configured to display data provided by the optical sensor 26 for the user. The display 29 may also be configured to display system parameters of the intubation device 10, such as power level of the power source, positions and/or velocities of the drive device(s) and/or actuation device(s), and/or connectivity/disconnectivity of the intubation device 10 to an embedded or external data receiving and processing unit and/or an embedded or external controller.

The intubation device 10 may comprise a controller having computational circuitry configured to operate the intubation device 10 in an automatic mode and/or a manual mode.

The controller may process data received from the optical sensor 26 to determine a location and/or orientation of at least a section, preferably a distal section or a distal tip, of the laryngoscope blade 18 and/or the endotracheal tube 24 and/or the stylet 20 relative to an anatomy of the patient proximate the trachea of the patient.

The embodiment shown in FIG. 6 demonstrates that the sled 14 may alternatively comprise other elements, such as sliders, gears, rollers, belts, or wires configured to engage at least a portion of the guide member 12 to translationally move the sled 14 in the advancing direction, and preferably also in a direction opposite to the advancing direction.

As shown in FIG. 6, the sled 14 may have a device drive 30 configured to drive the sled via belts or cables 31. The drive device 30 is arranged on or in the handle 16 according to the embodiment shown in FIG. 6. Alternatively, the drive device 30 may be located at least partially in and/or on the guide member 12 and/or sled 14 and/or handle 16. The belts or cables 31 may be anchored in the sled 14 and/or the guide member 12. The belts or cables 31 may be oriented by a plurality of idler wheels 32.

FIG. 7 shows a process that may be used by the intubation device 10 for completing the entire intubation procedure from intubation setup to device navigation to intubation. FIG. 7 shows actions and decisions/determinations of the intubation device, which are identified by rectangular boxes and rhombi, respectively, each having solid lines. FIG. 7 further shows user actions, which are identified by rectangular boxes having dashed lines.

After the user places the intubation device 10 in the patient, a computational circuitry provided in the intubation device 10 and/or provided externally may determine at 33 if the laryngoscope blade 18 is correctly place. If it has been determined that the laryngoscope blade 18 is not correctly placed, the computational circuitry may communicate with the user utilizing the display 29 and/or acoustic and/or haptic feedback to give instructions 34 to the user to adjust the positioning of the laryngoscope blade 18.

The computational circuitry may determine at 35 if the anatomy of the patient has been detected with at least one of the sensors 26. If the computational circuitry has detected the anatomy of the patient, the computational circuitry may communicate at 36 with the user utilizing the display 29 and/or acoustic and/or haptic feedback to demonstrate the detected anatomy and/or the calculated automated navigation. If the computational circuitry has not detected the anatomy of the patient, the computational circuitry may communicate at 37 with the user, e.g., via the display and/or acoustic and/or haptic feedback, that no anatomy has been detected.

The user may choose to automatically and/or manually drive the navigation of the bending section 22 and/or the sled 14 by using the manual input devices 28. The device may drive at 38 the manipulating mechanism to drive the bending section 22 and/or the drive device 30 to drive the sled 14 based on the input from the manual and automated modes.

The computational circuitry may determine at 39 if the navigation has successfully reached the target anatomy.

In a manual mode, the controller may then compare the determined location and/or orientation with manual input commands received by one of the manual input devices 28 arranged on the user interface.

The controller may then send electrical commands to drive devices and/or actuation devices of the intubation device 10, e.g., the drive device 30 configured to drive the sled 14 and/or the actuating device configured to actuate the manipulating mechanism to manipulate the stylet 20.

In an automatic mode, the controller may automatically, i.e., without user intervention, or at least reduced user intervention vis-à-vis a full manual mode, via the manual input devices 28, send electrical commands to drive devices and/or actuation devices of the intubation device 10, e.g., the drive device 30 configured to drive the sled 14 in an advancing direction and/or the actuating device configured to actuate the manipulating mechanism to manipulate the stylet 20.

The controller may be housed in the sled 14 or in the handle 16. In case the controller is arranged in the handle 16, the controller may be configured to communicate wirelessly and/or via communication cables with the sled 14, e.g., with a drive device and/or actuation device housed in the sled 14.

An external controller, i.e., not embedded in the intubation device 10, may also be provided.

The intubation device 10 may also be configured to communicate, preferably wirelessly, with an external data receiving and processing unit (not shown), such as a smartphone, a tablet, or a pc.

Thus, the intubation device 10 may transfer data, for instance data provided by the optical sensor 26, to the external data receiving and processing unit, preferably via a transmitting unit arranged on or in the intubation device 10.

Preferably, the external data receiving and processing unit is configured to process the data and provide information, e.g., visual information, to the user, e.g., via a display of the external data receiving and processing unit. In this case, the display 29 can be maintained as a component of the intubation device 10 as a primary or secondary source of visual information. However, the display 29 can also be omitted in this case and the display of the external data receiving and processing unit may be the only source of visual information for the user.

The external data receiving and processing unit may be embedded in an external controller, such as the external controller described above.

The intubation device 10 may also be configured to communicate with external data sending devices such as other medical equipment including, but not limited to, a pulse oximeter and/or an electrocardiogram. The display 29 may be used for showing the system parameters and sensor variables from the external data sending devices. Thus, the user may monitor critical patient parameters on the display 29 of the intubation device.

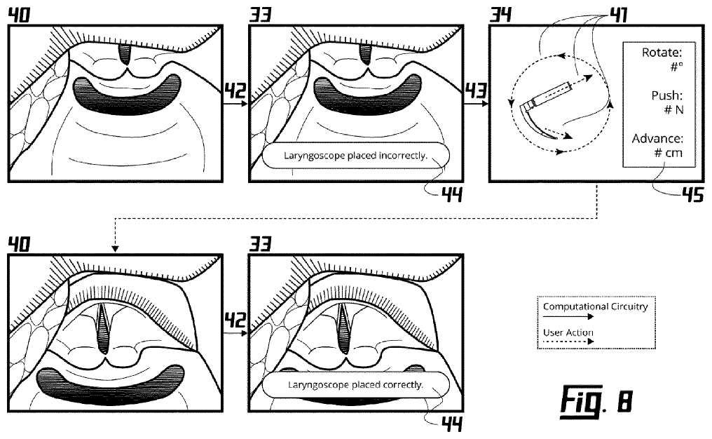

FIG. 8 shows a graphical user interface and a mode of operation of the intubation device 10. The graphical user interface may be provided on a screen of a component of the intubation device 10 and/or on a screen of an external computing device, such as a personal computing device of the user. In FIG. 8, action provided and/or performed by the computational circuitry (“Computational Circuitry”) is indicated by a solid arrow, whereas action(s) provided and/or performed by the user/practitioner (“User Action”) is/are indicated by a dashed arrow, respectively. FIG. 8 shows an image 40 of one or more anatomical features of the patient during operation of the intubation device 10, e.g., as the laryngoscope blade 18 is being inserted into or towards the patient's pharyngeal cavity, which may be provided by one or more optical sensors, preferably video sensors, of the intubation device 10, e.g., the sensor 26 shown in FIGS. 1 to 6. Preferably the image 40 is a sequence of images and/or a live video feed. The optical sensor(s) may be arranged on the stylet 20, preferably at a distal tip or distal section thereof, and/or on the laryngoscope blade 18, preferably at a distal tip or distal section thereof. The optical sensor may be a fiberoptic or any digital image sensor including, but not limited to, CMOS and CCD.

The image 40 may be displayed on a display screen which is visible by the user. The computational circuitry may be configured to process and/or analyze 42 the image 40 to determine if the intubation device 10, e.g., the laryngoscope blade 18, has been placed correctly, as indicated at 33 in FIG. 8. In order to determine that the intubation device 10, e.g., the laryngoscope blade 18, has been placed correctly, the computational circuitry may be configured to determine if the laryngoscope blade 18 has reached a target position, preferably a predetermined target position, relative to the patient's anatomy and/or if the laryngoscope blade 18 is within a certain, preferably predetermined, range of the target position. Alternatively, or additionally, the computational circuitry may be configured to determine if the laryngoscope blade 18 has reached a target orientation, preferably a predetermined target orientation, relative to the patient's anatomy and/or if the laryngoscope blade 18 is within a certain, preferably predetermined, range of the target orientation. If the computational circuitry determines that the laryngoscope scope 18 has been placed correctly, e.g., if the laryngoscope blade 18 has reached or is within a range of the target position and/or a target orientation relative to the patient's anatomy, the computational circuitry may be configured to communicate 44 the correct placement of the laryngoscope blade 18 to the user, e.g., visually, acoustically and/or haptically. The user may then begin inserting the endotracheal tube 24 into the patient's trachea.

The computational circuitry may be configured to determine at least one deviation between a current position and/or a current orientation of the laryngoscope blade 18 relative to the patient's anatomy and a target position, preferably a predetermined target position, and a target orientation, preferably a predetermined target orientation, respectively, relative to the patient's anatomy.

The computational circuitry may be configured to process and/or analyze 43 the image 40 to determine one or more maneuvers 41, e.g., one or more translational movements and/or one or more rotational movements of a section of the intubation device 10, e.g., the laryngoscope blade 18, e.g., in order to move the intubation device 10, e.g., the laryngoscope blade 18 to, or at least towards, a target position and/or a target orientation and/or within a range of the target position and/or the target orientation.

If the computational circuitry determines that the intubation device 10, e.g., the laryngoscope blade 18, has not been placed correctly, e.g., if the laryngoscope blade 18 has not reached and/or is not within a certain range of a target position and/or a target orientation relative to the patient's anatomy, the computational circuitry may be configured to communicate 44 the incorrect placement of the laryngoscope blade 18 to the user, e.g., visually, acoustically and/or haptically. The intubation device 10 may also be configured to provide information related to the one or more maneuvers 41 determined by the computational circuitry to the user, e.g., visually, acoustically and/or haptically, so that the user may follow the guidance of the intubation device 10 by performing the maneuvers 41 determined by the computational circuitry.

For instance, the intubation device 10 may be configured to provide, e.g., communicate, one or more instructions to the user relating to the maneuvers 41, e.g., via a schematic, preferably animated, representation of the maneuvers 41 and/or via specific values or value ranges 45, such as a degree of rotation of the intubation device 10 and/or the laryngoscope blade 18 in [*], a force in [N] to be exerted on the intubation device 10 and/or the laryngoscope blade 18, preferably against the patient's tongue, and/or a degree of advancement or retraction of the intubation device 10 and/or the laryngoscope blade 18 in [cm] further into the patient's body or further outward of the patient's body, respectively, which may be displayed on one or more screens visible by the user.

1-15. (canceled)

16. An intubation device for delivering an endotracheal tube to a trachea of a patient, the intubation device comprising:
at least one laryngoscope blade for providing access to a pharyngeal cavity of the patient;
at least one guide member which is curved in at least a section thereof;
at least one sled configured to be coupled to the endotracheal tube and advance the endotracheal tube to the trachea of the patient; and
at least one drive device configured to drive the sled in an advancing direction;
wherein the sled is movably coupled to the guide member such that the sled is translationally movable along the guide member relative to the guide member in the advancing direction.

17. The intubation device according to claim 16, wherein the guide member is substantially rigid.

18. The intubation device according to claim 16, comprising at least one stylet coupled to the sled, wherein the stylet comprises at least one bendable section configured to be bent in at least one degree of freedom or at least two degrees of freedom or at least three degrees of freedom.

19. The intubation device according to claim 18, wherein the endotracheal tube is configured to be positioned at least partially over and follow the shape of the stylet.

20. The intubation device according to claim 18, wherein the stylet is configured to manipulate at least a section of the endotracheal tube by manipulating at least a section of the stylet.

21. The intubation device according to claim 18, comprising a manipulating mechanism configured to manipulate at least a section of the stylet, wherein the manipulating mechanism comprises at least one angulation wire connected to the manipulating mechanism and extending at least partially through the stylet, wherein the angulation wire is configured to bend the stylet at the bendable section in at least one degree of freedom.

22. The intubation device according to claim 21, comprising at least one actuating device configured to actuate the manipulating mechanism.

23. The intubation device according to claim 22, wherein the at least one actuating device is housed in the sled or in a handle of the device.

24. The intubation device according to claim 16, comprising a controller configured to automatically drive the sled.

25. The intubation device according to claim 24, wherein the controller is configured to automatically drive the sled based on visual data provided by an optical sensor

26. The intubation device according to claim 18, comprising a controller configured to automatically manipulate the stylet.

27. The intubation device according to claim 26, wherein the controller is configured to automatically manipulate the stylet based on visual data provided by an optical sensor.

28. The intubation device according to claim 16, comprising a user interface having at least one manual input device configured to receive manual input commands from a user, wherein the manual input device comprises at least one button or at least one switch or at least one joystick for receiving manual input commands from a user.

29. The intubation device according to claim 28, wherein the manual input commands comprise at least one of navigation commands for manually advancing the sled, stylet manipulating commands for manually manipulating at least a section of a stylet that is coupled to the sled and comprises at least one bendable section, and automation activation commands for activating or deactivating an automatic mode of the intubation device in which the sled is driven automatically or the stylet is manipulated automatically.

30. The intubation device according to claim 16, wherein the guide member is a toothed rack which is curved in at least a section thereof, wherein the sled comprises a gearwheel driven by the drive device, wherein the gearwheel is configured to engage at least a portion of the guide member to translationally move the sled in the advancing direction.

31. The intubation device according to claim 16, wherein the sled or the guide member or a handle of the device comprise a pulley wheel driven by the drive device, wherein the pulley wheel is configured to engage a cable anchored on the guide member or the sled or the handle, wherein the cable may be oriented by a plurality of idler wheels.

32. The intubation device according to claim 16, comprising a remote controller configured to drive the sled or manipulate a stylet that is coupled to the sled and comprises at least one bendable section, wherein the controller is configured to drive the sled or manipulate the stylet based on a remote user providing navigation commands based on visual data provided by an optical sensor.

33. An intubation device for providing assistance to a user in delivering an endotracheal tube to a trachea of a patient, the intubation device comprising:
at least one laryngoscope blade for providing access to a pharyngeal cavity of the patient;
at least one sensor configured to capture the location or orientation of at least a section of the laryngoscope blade or the endotracheal tube relative to an anatomy of the patient; and
computational circuitry configured to determine if the laryngoscope blade has been correctly positioned, wherein the intubation device is configured to instruct the user how to correctly place the laryngoscope blade based on data provided by the sensor.

34. The intubation device according to claim 33, wherein the at least one sensor is configured to capture the location and orientation of at least a section of the laryngoscope blade or the endotracheal tube relative to an anatomy of the patient.

35. The intubation device according to claim 33, wherein the at least one sensor is configured to capture the location or orientation of a distal section or a distal tip of the laryngoscope blade or the endotracheal tube.

36. The intubation device according to claim 33, comprising at least one stylet which includes at least one bendable section configured to be bent in at least one degree of freedom or at least two degrees of freedom or at least three degrees of freedom.

37. The intubation device according to claim 36, wherein the endotracheal tube is configured to be positioned at least partially over and follow the shape of the stylet.

38. The intubation device according to claim 36, wherein the stylet is configured to manipulate at least a section of the endotracheal tube by manipulating at least a section of the stylet.

39. The intubation device according to claim 36, comprising:
a manipulating mechanism configured to manipulate at least a section of the stylet, wherein the manipulating mechanism comprises at least one angulation wire connected to the manipulating mechanism and extending at least partially through the stylet, wherein the angulation wire is configured to bend the stylet at the bendable section in at least one degree of freedom.

40. The intubation device according to claim 36, comprising:
a controller configured to automatically manipulate the stylet.

41. The intubation device according to claim 40, wherein the controller is configured to automatically manipulate the stylet based on visual data provided by the sensor.

42. The intubation device according to claim 36, wherein the sensor is an optical sensor arranged at a distal tip of the stylet, the optical sensor being configured to provide visual data of an anatomy of the patient.

43. The intubation device according to claim 33, wherein the sensor is an optical sensor arranged on a portion of the laryngoscope blade, the optical sensor being configured to provide visual data of an anatomy of the patient.

44. The intubation device according to claim 33, wherein the sensor is a camera.

45. A kit comprising an intubation device according to claim 1 and an external data receiving and processing unit communicatively connectable to the intubation device and configured to receive and process data received by the intubation device.

46. The kit according to claim 45, wherein the external data receiving and processing unit is configured to send data to the intubation device.
